(19) United States
(12) Patent Application Publication
(10) Pub. No.: US 2003/0109903 A1
(43) Pub. Date: Jun. 12, 2003

(54) Low profile subcutaneous enclosure

(75) Inventors: Peter G. Berrang, Victoria (CA); Henry V. Bluger, Victoria (CA); Stacey D. Jarvin, Brentwood Bay (CA); Bradley J. D. Penner, Victoria (CA)

Correspondence Address: Paul Smith, Paul Smith Intellectual Property Law, 1508 West Broadway - Suite 330, Vancouver, BC, V6J 1W8, CA

(73) Assignee: Epic Biosonics Inc.

(21) Appl. No.: 10/012,341

(22) Filed: Dec. 12, 2001

(51) Int. Cl. A61N 1/375

(52) U.S. Cl. 607/36

(57) ABSTRACT

A design and method for fabricating a low profile, biocompatible, hermetically sealed, subcutaneous enclosure for human implants is disclosed. The implant includes bonded top and a bottom plates, and an insert bonded to an aperture in the bottom plate. Passageways that extend through the insert are filled with a conductive material to provide hermetic but conductive leads to the interior of the implant. Successive layers of metals are used to bond the insert to the bottom plate and to establish a bond between a first layer of polymer covering the insert and bottom plate. An additional polymer layer containing contact pads and conductor lines is bonded to the first layer. The implant includes a low profile safety on-off switch and a hermetically sealed low profile rechargeable battery.

### BACKGROUND OF THE INVENTION

Only certain materials can be permanently implanted in humans without triggering unacceptable tissue response. Numerous studies have been conducted to identify the biocompatibility of various implant materials (see for example “Biocompatibility of Clinical Implant Materials”, Volumes 1 and 2, edited by David F. Williams, published by CRC Press, Inc., Boca Raton, Fla., USA). Some commonly used biomaterials include titanium (and some alloys thereof), platinum, tantalum, niobium, iridium, gold, some ceramics (such as pure alumina), certain carbon materials, some silicones, and polymers such as the fluorocarbons FEP, PTFE, PVDF, PFA, PCTFE, ECTFE, ETFE and MFA (a copolymer of TFE and PVE), polyethylene's, polypropylenes, polyamides and polyimides. Those skilled in the art will appreciate that the chemical purity, form, location, mechanical stress and loading, and actual use of these materials within the body also dictate the overall tissue response and acceptability of such materials or combinations thereof. Although a material may be considered biocompatible (or bio-inert) for a particular implant application, such a material may not be hermetic, where hermetic is a general term used to describe the permeability of the material to fluid transfer or gas diffusion. Organic based compounds, including, for example, polymers and epoxies are not considered hermetic over long periods of time, as evidenced, for example, by the early models of pacemakers encapsulated in epoxy which failed after several years due to the slow diffusion of water into the electronics. To achieve a level of hermeticity acceptable for implant use, an enclosure must be comprised of biocompatible metal, ceramic, glass, or combinations thereof.

Many implant enclosures require that electrical leads enter from outside the hermetic enclosure to inside the hermetic enclosure. Those skilled in the art will appreciate that there are various methods to achieve a plurality of insulated electrical connections from outside a hermetic enclosure to inside a hermetic enclosure, while still maintaining the enclosure's hermeticity. Such methods include high temperature fusion bonding, brazing, vacuum sputtering and laser welding. (see for example “Cochlear Implant Design and Construction” by S. J. Rebscher, published in Chapter 4, Cochlear Implants, Ed. by Roger F. Gray, Croon Helm, London, 1985). However, such prior art methods are generally bulky, and space consuming, and are not always suitable, especially for head-mounted subcutaneous prostheses requiring low profile, small footprint enclosures and with large numbers of electrical connections.

In certain parts of the body where device size is not critical, prior art “bulky” implants such as pacemakers, incontinence stimulators and heart pumps can be reasonably positioned within the body. These prior art implants also require relatively few electrical lead-through connections. However, devices such as cochlear implants, especially fully implantable cochlear implants as described in U.S. patent application Ser. No. 09/450,025, are preferably mounted on the head for surgical reasons. Such head-mounted implants should have a relatively low profile and small geometric area since the thickness of the skin/muscle tissue overlying the skull and the curved shape of the skull, especially in infants, set practical limits on the implant's diameter and thickness. Additionally, totally implantable cochlear implants may require 30 or more electrical lead-throughs from outside the enclosure to inside the enclosure, which connections must not significantly impact the enclosure's low profile or small geometric area requirement.

Communication with a totally subcutaneous implant from outside the body is generally accomplished via a transcutaneous RF link. However, in the event that the RF link fails, or in the event that the external RF link device is not readily available, it is prudent from a safety perspective to have available an external mechanically-actuated on-off switch. Such mechanical back-up on-off switch is a paramount safety requirement in cases where the failure of the subcutaneous implant creates a potential danger to the implantee. For example, in the event of the sudden failure of a totally implanted cochlear implant, the implantee must have the ability to immediately, by mechanical means, switch off electrical power to the stimulation electrodes to prevent neural damage and or pain. U.S. patent application Ser. No. 09/450,025, included herein by reference, describes various means for a mechanically actuated switch, using a piezo crystal, PVDF piezoelectric film or mechanically induced actuation. The piezo type switches tend to be relatively bulky, and fragile, especially for sudden impact loading. Also, the voltage output from piezo crystals generally requires signal conditioning to be effectively used as a high reliability independent on-off switch. The use of PVDF film switches requires the film to be held in a “drum like” configuration to obtain maximum sensitivity, which fabrication is difficult. The PVDF film is also very fragile. The present invention addresses the need for a simple, highly reliable, low profile safety on-off switch that can be housed within a biocompatible, hermetic enclosure and actuated mechanically from outside the body.

Finally, the prior art does not address the requirement for a low profile, safe, high cycle, rechargeable battery housed within a hermetic enclosure. Although U.S. patent application Ser. No. 09/450,025 describes the use of a solid state lithium chemistry battery for use in a totally implantable cochlear implant, such a battery design uses a highly reactive lithium anode, which therefore dictates that the battery be independently hermetically sealed against moisture and gases such as oxygen and nitrogen. Accordingly, there is a need to independently hermetically encapsulate said solid state lithium battery using a very low profile hermetic sealing process, which unique sealing process is disclosed herein.

The present invention seeks to overcome some or all of the foregoing limitations of the prior art.

### SUMMARY OF THE INVENTION

To meet the requirements of a low profile subcutaneous prosthesis, the present invention comprises a compact enclosure that is hermetic, biocompatible, and has a plurality of low profile, high density electrical interconnections between the enclosure's inside and outside. The enclosure also contains a low profile, reliable, manually activated (i.e. interactive) on-off safety switch, and a low profile hermetically encapsulated high charge cycle rechargeable battery. Conventional low profile microelectronics circuits can also be contained within the enclosure, which enclosure is preferably comprised of thin-walled laser welded titanium or titanium components.

According to one aspect of the invention, the enclosure comprises a top plate and a bottom plate secured to the top plate to form the enclosure. The bottom plate includes an aperture that is filled by a non-conducting insert through which are formed a number of passageways which are then filled with conductive material. There is thereby established a hermetic electrical connection from outside the enclosure to inside the enclosure.

A first polymer layer, preferably a fluoropolymer such as FEP, is bonded to the bottom plate or to the insert or both. The first polymer layer is fused with a polymer O-ring anchor formed into a retaining groove about or near the perimeter of the bottom plate and/or insert.

A second polymer layer is bonded to the first layer and includes conducting wires and bonding pads connected to the conducting material in the sealed passageways.

The second polymer layer may be protected by the addition of a metal foil, which foil is preferably comprised of titanium.

A third polymer layer may be bonded to the second polymer layer, preferably by heating in a vacuum, to act as a protective layer. In an alternate embodiment, such third polymer layer may be further protected by the addition of a metal foil, which foil is preferably comprised of titanium.

In a further embodiment, the first, second and third polymer layers can also be comprised of one or more of fluorocarbons (other than FEP), polypropylene, polyethylene, polyimide, or polyamide.

A manually activated on-off switch is mounted on the top plate by simply pushing against the outside of the skin overlaying said enclosure.

A rechargeable (or secondary), numerous charge cycle, safe, low profile hermetically sealed battery is provided that can be recharged from outside the body by using an RF inductive link.

A low profile receiver coil may be attached to the enclosure. The coil comprises one or more turns of conductive material encapsulated in a polymer film, such coil used as an RF inductive link to communicate with an outside-the-body coil to enable recharging of said battery and as a means to electronically communicate with electronics contained within the enclosure.

Conventional electronic components are provided inside the enclosure, with the top plate preferably laser welded to the bottom plate, and an inert leak detection gas such as helium contained within said enclosure to determine the enclosure's hermeticity.

Other specific aspects of the invention are detailed in the claims, which are incorporated in their entirety in this summary be reference. Yet further aspects of the invention will be appreciated by reference to the detailed description of the invention that follows.

### BRIEF DESCRIPTION OF THE DRAWINGS

The preferred and alternative embodiments of the invention will be described by references to the accompanying drawings, in which:

FIG. 1 is a cross sectional sketch showing the preferred embodiment of the invention.

FIG. 2 is an isometric sketch of the implant adapted for use in a totally implanted cochlear implant.

FIG. 3 is a lateral view of the left side of the head showing the implant adapted for use in a totally implanted cochlear implant, in place, said view also illustrating one embodiment of an incision on the head to gain access for implantation.

FIG. 4 is a lateral view of the left side of the head illustrating a method for manually activating the safety on-off switch.

FIG. 5 is a cross sectional view of an embodiment of the bottom plate, with the non-conductive insert attached, and the passageways within said insert filled with a metal or alloy, and the distal surface of said bottom plate coated with two layers of metal.

FIG. 6 is a cross sectional view of an embodiment of the bottom plate including a composite ring positioned for attachment to the distal metal layer on said bottom plate.

FIG. 7 is a cross sectional view of an embodiment of the bottom plate showing the metal layers on a composite ring fused to the bottom plate.

FIG. 8 is an enlarged cross sectional view of the plurality of layers between the polymer layer and the distal surface of the bottom plate shown in FIG. 7.

FIG. 9 is a cross sectional view of an embodiment of the bottom plate wherein metal layers on the composite ring have been chemically etched in a pattern of a plurality of “wavy” concentric circles.

FIG. 10 is a view taken along line A-A of FIG. 9.

FIG. 11 is a cross sectional view of an embodiment of the bottom plate.

FIG. 12 is a cross sectional view of the preferred embodiment of the bottom plate, with the non-conductive insert attached, and the passageways within said insert filled with metal or alloy, and the distal surfaces of both said bottom plate and said insert polished to form a single contiguous flat surface.

FIG. 13 is a cross sectional view of an embodiment of the bottom plate.

FIG. 14 is a view taken along line B-B of FIG. 13.

FIG. 15 is a cross-sectional view of an embodiment of the bottom plate and insert.

FIG. 16 is an enlarged cross sectional view of part of the plurality of layers between the first and second polymer layers, and the distal surface of the bottom plate, and the non-conducting insert, shown in FIG. 15.

FIG. 17 is a cross sectional view of an embodiment of the bottom plate.

FIG. 18 is a cross sectional view of an embodiment of the bottom plate including a ring of a (first) polymer material positioned for attachment to the heat treated distal surface on the bottom plate.

FIG. 19 is a cross sectional view of an embodiment of the bottom plate showing the ring of a (first) polymer layer, directly fusion bonded, in a vacuum, to the distal surface of the bottom plate.

FIG. 20 is a cross sectional view of an embodiment of the bottom plate.

FIG. 21 is a cross sectional view of the top plate and safety on-off switch of the preferred embodiment of the invention.

FIG. 22 is a cross sectional subcutaneous view of the preferred embodiment of the invention, with the on-off switch being activated by pressing a finger against the skin overlaying the proximal part of the implant enclosure.

FIG. 23 is a cross sectional view of the top plate and the safety on-off switch of an alternate embodiment of the invention.

FIG. 24 is a cross sectional view of the encapsulated battery.

FIG. 25 is a cross sectional view of an alternate embodiment of the invention containing a bio-inert coating substantially around the invention enclosure.

### DETAILED DESCRIPTION OF THE PREFERRED AND ALTERNATIVE EMBODIMENTS OF THE INVENTION

For clarity, the authors define proximal and distal as viewed externally by an observer looking at the invention implanted in the implantee. Thus, that part of the implant facing towards the outside of the body is “proximal”, and that part of the implant facing towards the inside of the body is “distal”.

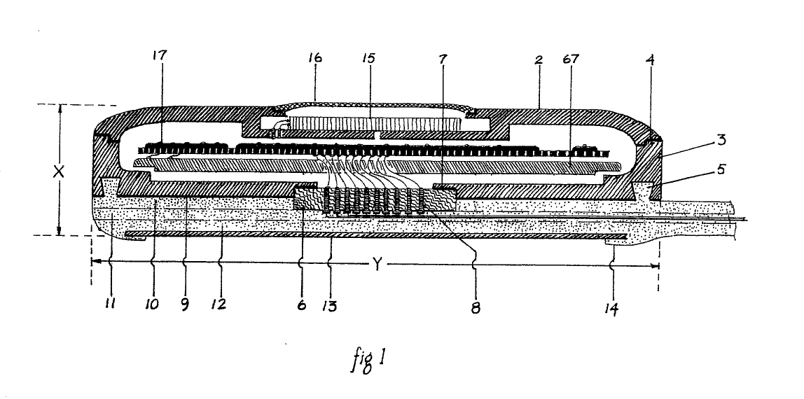

FIG. 1 illustrates the preferred embodiment of the implant according to the invention, depicting the salient components, where said components interact to achieve a novel overall low profile, subcutaneous enclosure. The implant is preferably round, but alternatively may be square, rectangular or any other shape. It is designed to be implanted subcutaneously, preferably on the head with minimum excavation of the skull bone and with minimum impact to the blood supply to the skin overlying the implant. Accordingly, the height ‘X’ and diameter ‘Y’ shown in FIG. 1 are designed to be minimal, i.e. about 2-7 mm, preferably about 4-6 mm, for dimension ‘X”, and about 3-50 mm, preferably about 20-30 mm, for dimension ‘Y’.

The implant includes a top plate 2 and a bottom plate 3, which can be made of one or a combination of bio-compatible metals such as titanium, tantalum, niobium, iridium, gold or alloys thereof, or glasses and ceramics (such as alumina or sapphire), but that are preferably made of titanium or an alloy of titanium. Plates 2 and 3 can be laser welded at location 4 as a final assembly step. It should be noted that the term “plate” used throughout this disclosure and in the claims is defined to include any generally planar, three-dimensional shape that is machined or formed. An aperture is provided in the bottom plate, preferably in the central portion of the bottom plate.

The retaining groove 5, shown for illustrative purposes as a truncated “V” shape around the circumference of the distal side of the bottom plate 3, is filled with a polymer, preferably the fluoropolymer FEP, which retaining feature acts as a strong anchor to hold, via fusion bonding, subsequent fluoropolymer layers 9, 10, 11 and 12. Such a retaining groove is designed to enclose a non-conducting insert 6 that is secured in the aperture in the bottom plate 3, by securing layers of polymer that are formed over the insert. The polymer filling the groove 5 acts as an O-ring anchor. Once the groove is filled and a polymer film layer is pressed and heated against the O-ring, the interface between them will melt, seamlessly fusing the O-ring and the polymer layer. Those skilled in the art will note that a myriad of possible configurations are possible to create a retaining groove, which alternate shapes are within the intended scope of this invention.

Non-conductive insert 6, which can be shaped substantially round, oval, square or rectangular can be comprised of glass or ceramic (such as alumina or sapphire), is preferably comprised of alumina.

The insert 6 includes a plurality of passageways extending therethrough. The insert can be bonded to a shoulder or flange formed about an aperture in the bottom plate 3, using a metal or alloy preferably comprised of gold, or an alloy of gold, or alternately silver, or an alloy of silver. In a further embodiment, nickel or an alloy of nickel, or an active brazing alloy containing titanium, for example, 1.75% titanium, 35.25% copper, and 63% silver can be used. Similarly, an electrically conducting metal or alloy comprised of titanium, silver, copper, gold, tin or any combination thereof, can be used to hermetically fill and seal the plurality of passageways in insert 6.

The filled passageways now also act as an electrical conduction path through the passageways in insert 6. Alternately, other alloys, solders or metals which bond hermetically to ceramics (or glasses) can be used to fill and seal the passageways. The diameter of the passageways are each about 50-1,000 microns, preferably about 300-500 microns, with the thickness of insert 6 about 100-1,000 microns, preferably about 250-500 microns, and the diameter of the insert being about 5-15 mm, preferably about 8-10 mm. Such dimensional sizes enable about 10-100 passageways, preferably about 20-40 passageways to be formed through insert 6, and hermetically sealed with an (electrically conductive) metal or alloy.

A feature of the implant is the process for bonding two biocompatible materials, namely an inert fluoropolymer (such as FEP), shown as layer 10 in FIG. 1, to an (oxidized) titanium surface (shown on the distal side of bottom plate 3 and depicted as line 9), which interface must remain bonded over many decades of time while exposed to body fluids and minor body temperature fluctuation cycles. If any conducting salts or body fluids act to delaminate the bonded interface, i.e. via capillary action of body fluids, then the electrically conducting alloy 8 in the passageways in insert 6 would electrically short, such shorting representing a device failure. The details of the bonded interface between polymer 10 (hereinafter referred to as first polymer layer 10) and the titanium surface on the distal side of bottom plate 3 is described in detail below and in the drawings.

A second polymer layer 11, preferably FEP, contains contact pads and the conductor wires leading to any sensing or stimulating elements implanted in the body. Said layer 11, which is about 10-100 microns thick, preferably about 25-50 microns thick, can be conveniently fusion bonded to layer 10 by heating in a vacuum at about 270-300° C. The contact pads embedded within layer 11, and the attachment of said pads to alloy 8, is described in detail below. In one embodiment, a protective foil, comprised of a bio-inert metal, glass or ceramic, preferably titanium, or an alloy of titanium is bonded to layer 11. However, in the preferred embodiment, a third polymer layer 12, preferably FEP, which is about 50-1,000 microns thick, preferably about 250-500 microns thick, can be conveniently heat bonded to layer 11 (by heating in a vacuum at about 270-300° C.), and is included to provide mechanical strength and protection for the conductors and pads imbedded in layer 11, and in one embodiment, can also contain conductor wires leading to a subcutaneous RF coil. Protective foil 13, which can be of bio-inert glass, ceramic or metal, preferably titanium or an alloy of titanium, about 50-100 microns thick, is heat bonded to layer 12, and further held in place at its perimeter by a small polymer lip 14, which lip is a ring of FEP heat bonded at its perimeter to layer 12.

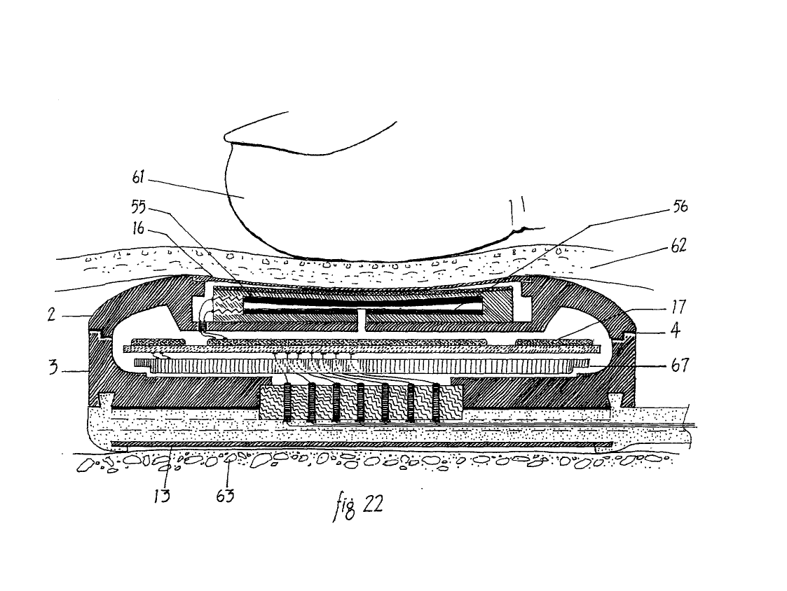

A mechanically actuated electrical contact switch 15 is incorporated into the proximal side of the implant 1, which switch 15 is covered with a flexible membrane 16, preferably titanium, about 25-250 microns thick, welded or fusion bonded under vacuum, at its circumference to the proximal side of the (titanium) top plate 2. By manually pressing membrane 16 with a finger (see FIG. 22), the switch contacts (shown in FIG. 22) are closed, and reopened when the pressure is released. Switch 15 is designed to withstand high impact loading. Details of said switch 15 are further described below.

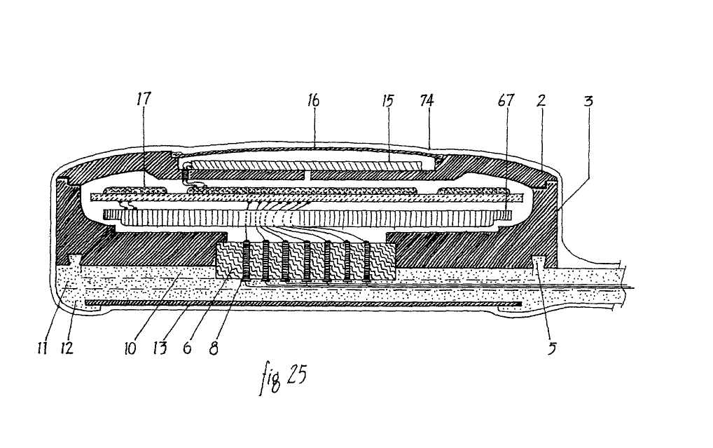

The implant according to the invention can also house low profile electronics, depicted by 17, and a hermetically sealed low profile battery 67 which is described in relation to FIG. 24.

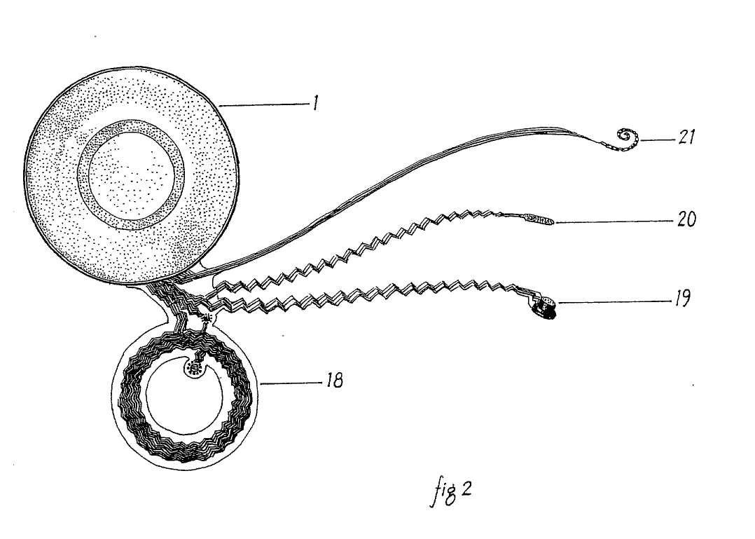

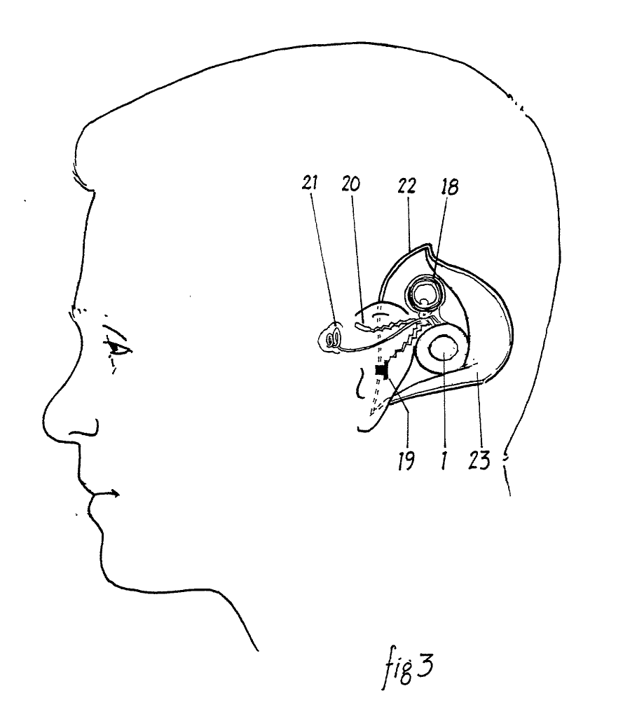

One application of the implant is for use in a totally implantable cochlear implant, which implant is illustrated in the isometric drawing shown in FIG. 2, which figure shows implant, RF inductive coupling coil 18, microphone 19, remote electrode 20 and electrode array 21. Said cochlear implant is shown mounted on the left side of the head in FIG. 3, which figure also shows surgical incision 22 and skin flap 23 pulled back, with the electrode array 21, microphone 19 and remote electrode 20 in position. FIG. 4 depicts a lateral view of the left side of the head, with the cochlear implant in place (subcutaneously), illustrating a method for manually activating the safety on-off switch by pressing, with finger 24, against the proximal surface of membrane 16 (shown in FIG. 1). For illustrative purposes, the skin covering the cochlear implant is not shown in FIG. 4.

FIG. 5 shows a cross sectional view of the preferred embodiment of bottom plate 3, with detail of the layers acting to hermetically bond insert 6 to bottom plate 3 using one or more metal or alloy layers as discussed above. The first layer 25 is preferably gold or an alloy of gold. Alternately, silver or an alloy of silver can be used. In a further embodiment, nickel or an alloy of nickel, or an active brazing alloy containing titanium can be used. The second metal layer 26 can be bonded to said first layer 25 using one or more of niobium, tin, bismuth, indium, zinc, gold or silver.

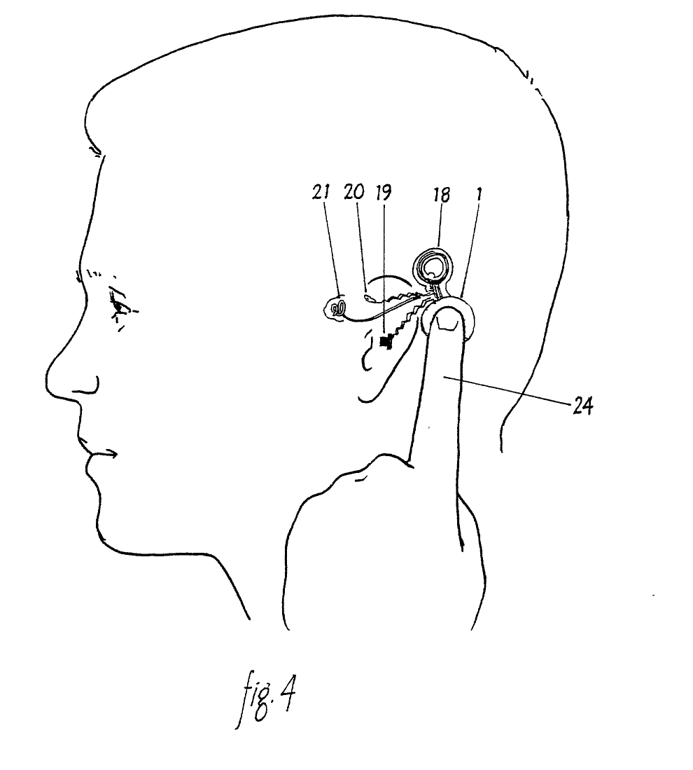

FIG. 6 is similar to FIG. 5, but also shows a composite ring 27, comprising a polymer 10 (preferably FEP) coated with multiple metal layers 28, positioned for attachment to the distal surface of alloy 26 on said bottom plate 3. Alternately, layers 25 and 26 (comprised of a metal or alloy such as gold, silver, or an active brazing alloy containing titanium) can also be adhered to the distal side of insert 6, so that composite ring 27 can then be attached to insert 6.

The bonding of composite ring 27 to alloy 26 is a non-trivial process, and represents a feature of the invention. The details of said bonding processes are herein described in detail.

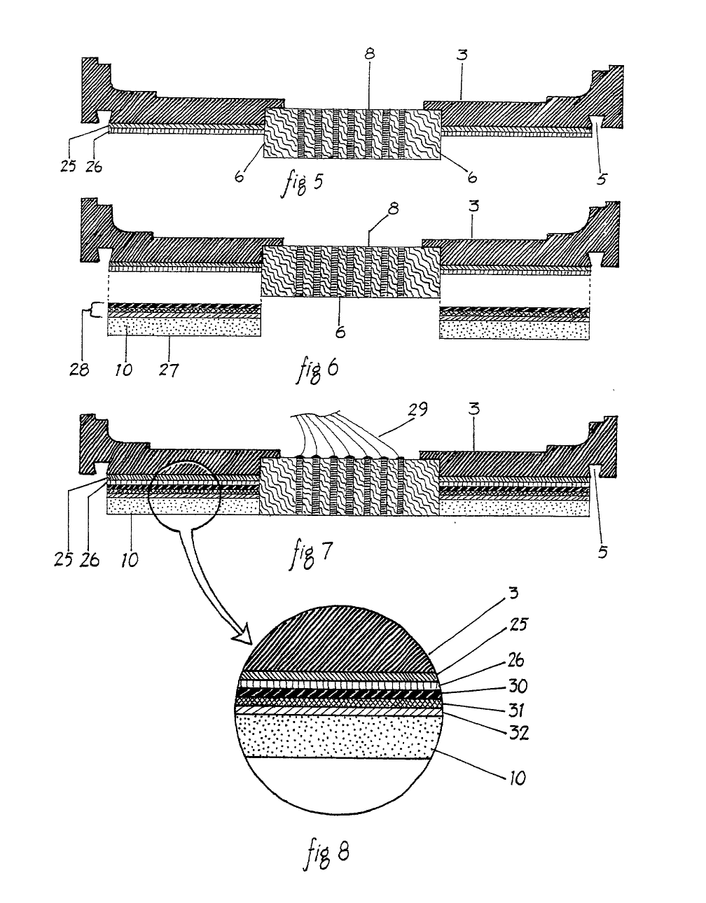

Referring to FIG. 6, multiple metal layer 28 comprises a bonded coating of one or more metal layers, preferably comprised of titanium 32, niobium 31 and gold 30, which layers are illustrated in better detail in FIGS. 7 and 8, where titanium layer 32 must firstly be bonded to first polymer layer 10. Additionally, tin (not shown) can be added to gold layer 30 and or layer 26. Preferably, multiple metal layer 28 is comprised of titanium 32, niobium 31, gold 30 and tin (not shown). Said bonding of titanium layer 32 to polymer 10 (i.e. FEP) is important, since it represents the key interface bond between a polymer and a metal (or alloy). Alternately, other metals that form oxides, such as zirconium, tantalum, aluminum, niobium and, to a lesser extent, tin, are alternative metals that can be used in place of titanium as the key bonded layer shown as layer 32. The addition of niobium 31, gold 30 and tin (not shown) over the titanium layer 32 are applied to be metal transitional layers to create compatible bonding layers that transition from titanium (on the polymer) to titanium (comprising bottom plate 3 or insert 6). In an alternate embodiment, the titanium layer 32 (adhered to polymer 10) can be directly bonded to another titanium surface (i.e. to titanium bottom plate 3 or to titanium coated insert 6) using silicone or epoxy. It should be noted that those skilled in the art will recognize that other metal transitional layer(s) other than niobium, such as zirconium, iridium, aluminum, tantalum and platinum can be used in place of niobium. Since polymer layer 10 has a maximum acceptable temperature for softening/melting, which in the case of FEP, is about 270-300° C., it is preferable to use alloys or metal systems (i.e. containing one or more of tin, silver, indium, tin or bismuth) with melting temperatures less than 300° C., or which can be vacuum deposited, i.e. via sputtering, vapor deposition or ion implantation at less than 300° C.

First polymer layer 10 has a thickness of about 10-500 microns, preferably about 25-50 microns, is highly inert and it is preferably chemically treated to make it more reactive, prior to bonding with titanium layer 32. Such activating treatment of FEP layer 10 is preferably done using a corona discharge, so as to add more polar chemical groups to the carbon-fluorine chain thereby making the FEP surface more reactive. In an alternate embodiment, the FEP surface can be treated with a solution of sodium based chemicals, such as sodium in liquid ammonia, sodium naphthalenide in tetrahydrofuran, sodium in napthalenes, diethylene glycol and dimethyl ether. In a yet further alternate embodiment, the FEP layer 10 may be treated with other chemical and electrical discharge methods to activate the surface to make it more reactive, and then using epoxies or other adhesives to bond to the FEP. The process for bonding metal layers titanium 32, niobium 31 and gold 30 and tin (not shown) to polymer layer 10 is as follows:

about 0.005 to 5 microns, preferably about 0.1-0.3 microns, of titanium is vacuum deposited onto corona or chemically activated FEP layer 10. During the vacuum deposition process, the titanium is in a non-oxidized state, and is thus highly reactive, and will securely bond to the treated FEP surface, creating a strong and intimate interface between the titanium layer and the FEP surface;

and, without breaking vacuum, depositing a layer of about 0.005-5 microns of niobium, preferably about 0.2-2.0 microns, over the titanium layer;

and, without breaking vacuum, depositing a layer of about 0.005-5 microns of gold, preferably about 1-3 microns, over the niobium layer;

and, without breaking vacuum, depositing a layer of about 0.005-5 microns of tin, preferably about 0.5-2 microns, over the gold layer.

FIG. 7 illustrates the low temperature metal or alloy layer 30 fusion bonded to low temperature alloy layer 26 (preferably tin) with electrical wires 29 attached to the proximal side of alloy 8 fused in the passageways in insert 6. Thus, composite ring 27 is now shown securely bonded to titanium bottom plate 3 using a myriad of only substantially corrosion resistant and biocompatible metal/alloy interface layers, namely, 25, 26, 30, 31, and 32 to accomplish this difficult task. FIG. 8 shows an expanded view of the various layers.

The above polymer-titanium bonding process, although intricate, provides an elegant and novel solution for securely bonding a polymer layer to a titanium surface, using substantially corrosion-resistant, biocompatible materials.

FIG. 9 shows an alternate embodiment for the process of bonding a polymer layer to a titanium surface. Said alternate embodiment addresses the issue of possible crack propagation of the (composite) deposited metal layer 28 (i.e. comprising titanium 32, niobium 31, 30 and tin—not shown) after they are deposited onto the (FEP) surface 10, said cracking and fracturing caused by the fact that FEP and other fluoropolymers have a high coefficient of thermal expansion. Such expansion coefficient causes significant thermal stresses in metal layer 28 during the FEP polymer layer fusion bonding steps, which occur at about 270-300° C. FIG. 9 shows a similar sketch as that shown in FIG. 5, but with the composite ring 33 now consisting of FEP layer 10 with rings 35 comprising a multiple metal layer. The metal layer rings consist of a plurality of concentric round, oval or “wavy” rings, preferably with said metal layer comprised of firstly, titanium 32, followed by niobium 31, gold 30 and tin (not shown). Gaps 34 have been chemically etched through said metal layer (to the FEP surface) creating said plurality of concentric “wavy” rings 35. The “wavy” rings 35 act to reduce tensile stress in layers 30, 31, 32 and tin layer (not shown) during thermal expansion of the underlying FEP layer 10. Essentially, any one contiguous “wavy” ring 35 represents an O-ring seal to prevent the incursion of body fluids between metal layer 26 and FEP layer 10 after heat bonding of layers 26 and 10.

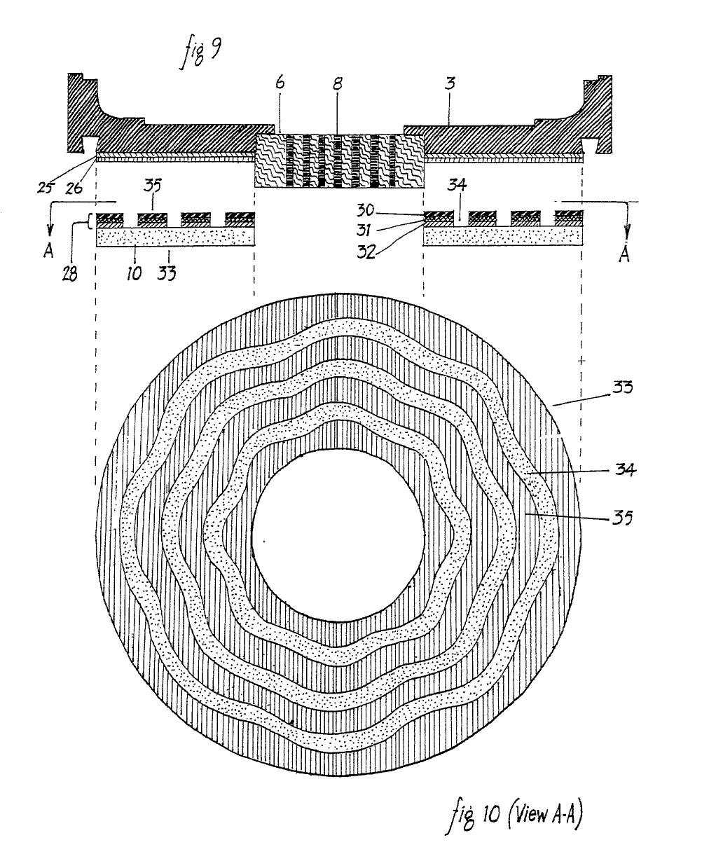

In an alternate embodiment, the wavy rings 35 could also be shaped to be substantially round, or oval, or any other contiguous shape. FIG. 10 is a top view of ring 33 and is depicted as view A-A.

FIG. 11 is similar to FIG. 9, but shows composite ring 33 bonded to alloy layer 26 to create a strong fluid-impermeable bond between polymer layer 10 and alloy layer 26. Even if one or more of the “wavy” rings 35 is severed due to thermal stress caused by the expansion/contraction of polymer 10 during any heat or lamination steps, the plurality of “wavy” rings creates a redundancy to ensure at least some rings 35 are intact.

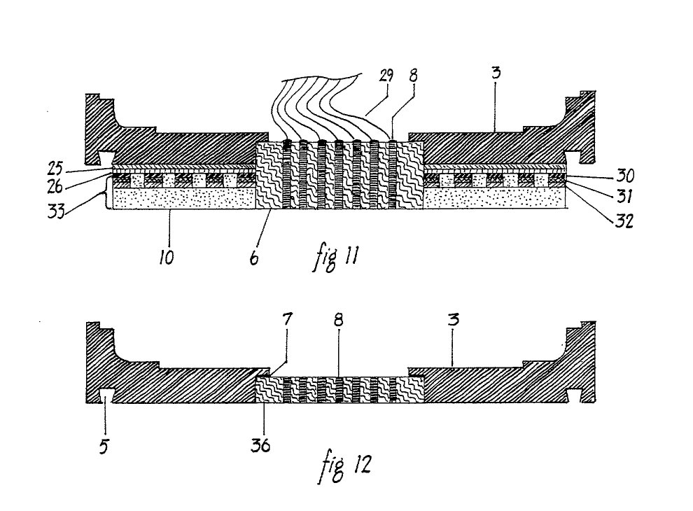

A further embodiment for creating a reliable fluid-impermeable bond is illustrated in FIGS. 12, 13, 14, 15 and 16. The objective of this embodiment is to create as large a sealing interface area between the polymer layer and the titanium surface as possible. FIG. 12 shows insert 36 fusion bonded to bottom plate 3 using a metal or alloy (such as gold, silver, nickel or a brazing alloy containing titanium), where the distal surfaces of bottom plate 3 and insert 36 are substantially flush. In an alternate embodiment, the size of the distal surface area of insert 6 can be made to be substantially larger than the distal surface area of bottom plate 3, such that most of the sealing interface occurs between the polymer layer and insert 6.

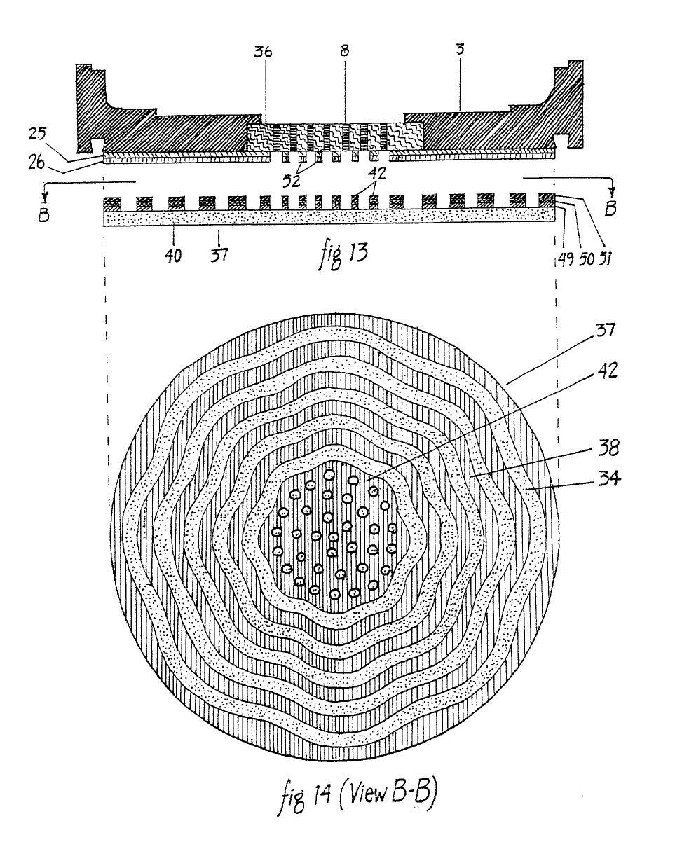

FIG. 13 depicts plate 3 with a non-conducting insert 36 bonded thereto, and a composite layer 37. Layer 25 and alloy layer 26 are bonded across the entire distal flush surfaces of insert 36 and plate 3, except around the passageways in insert 36, which passageways are hermetically sealed using an alloy 8, where layers 25 and 26 are not electrically connected to alloy 8 in said passageways. Composite layer 37, which layer is comprised of FEP layer 40 onto which is bonded a plurality of concentric etched “wavy” metal rings 38 (comprised of titanium 49, niobium 50, gold 51 and tin—not shown), except near the central part (shown in FIG. 14), where metal rings 38 are replaced by small “donut-shaped” rings 42, which rings 42 have a pattern to match similar “donut-shaped” rings 52 around passageways in insert 36. The inner diameter of rings 52 is larger than the alloy-filled passageways in insert 36 so that there is no electrical contact between the alloy-filled passageways in insert 36 and rings 52. FIG. 14 is a top view of composite layer 37 and is depicted as view B-B.

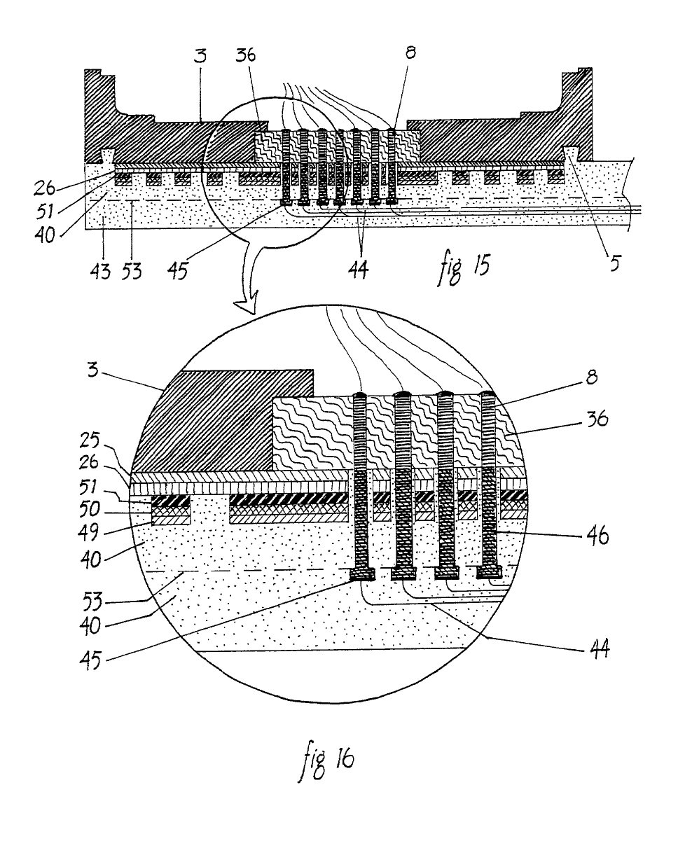

FIG. 15 shows gold layer 51 bonded to alloy layer 26 (preferably comprised of tin), with (FEP) polymer layer 43, containing conductor wires 44 and contact pads 45 fusion bonded to FEP layer 40. FIG. 16 shows an enlarged view of some of alloy 8 (used to hermetically seal the passageways in insert 36) electrically connected to contact pads 45 using preferably, a metal or alloy 46 comprising one or more of tin, bismuth, indium, zinc, silver and gold. Alternately, a conductive epoxy or paste can be used. Said metal or alloy 46 melts and forms an electrical connection between pad 45 (preferably fabricated from platinum) and alloy 8 during the fusion of FEP layers 40 and 43. Note that the FEP molded into retaining groove 5, during the fusion of layer 40 and layer 43, acts to mechanically anchor said fused FEP layers 40 and 43. Dashed line 53 separating layers 40 and 43 is for illustrative purposes only. During the bonding of layer 40 and 43, said layers both partially melt (or fuse) forming a seamless bond.

A further alternate embodiment for achieving a reliable fluid-impermeable bond between a polymer layer and titanium surface can be accomplished by directly fusion bonding the polymer layer to a bare titanium surface, the description of which is illustrated by reference to FIGS. 17, 18, 19 and 20. Referring now to FIG. 17, which depicts titanium bottom plate 3 fused to insert 6 with the passageways in said insert hermetically sealed with a conducting material (i.e. comprised of an alloy containing one or more of titanium, nickel, gold or silver). It should be noted that the surface of the titanium bottom plate 3 consists of a thin layer of TiO₂. To remove said TiO₂ layer is difficult, since such oxide layer will form in a few milliseconds when the bare titanium surface is exposed to normal atmosphere. Preferably, such oxide layer is removed or at least greatly thinned by heating the titanium base plate 3 at high temperature (i.e. about 500-700° C.) and high vacuum (i.e. about 10⁻⁵ to 10⁻¹⁰ torr) where such process substantially removes the oxide layer from the titanium surface to create a highly reactive titanium surface. Alternately, said titanium oxide layer may be thinned chemically by using a dilute solution of HF (hydrofluoric acid), preferably oxygen-outgassed, in a normal atmosphere, or preferably, an oxygen-free atmosphere (e.g. an argon dry box), and drying such surface in a normal atmosphere, or preferably, in an oxygen-free atmosphere. In a yet further embodiment, the titanium oxide layer may be removed mechanically by abrasion or machining in an inert atmosphere, such as an argon atmosphere.

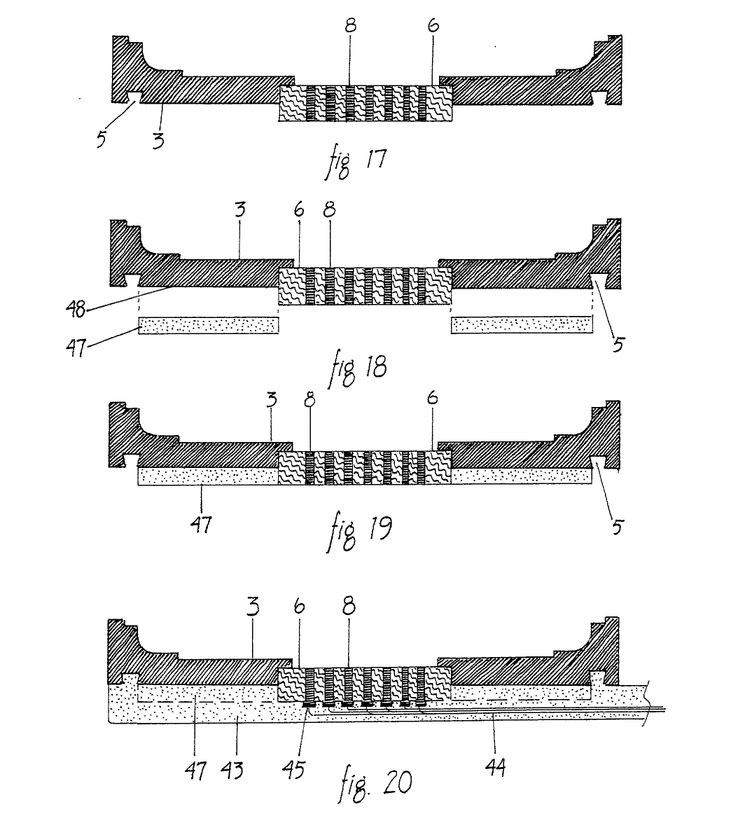

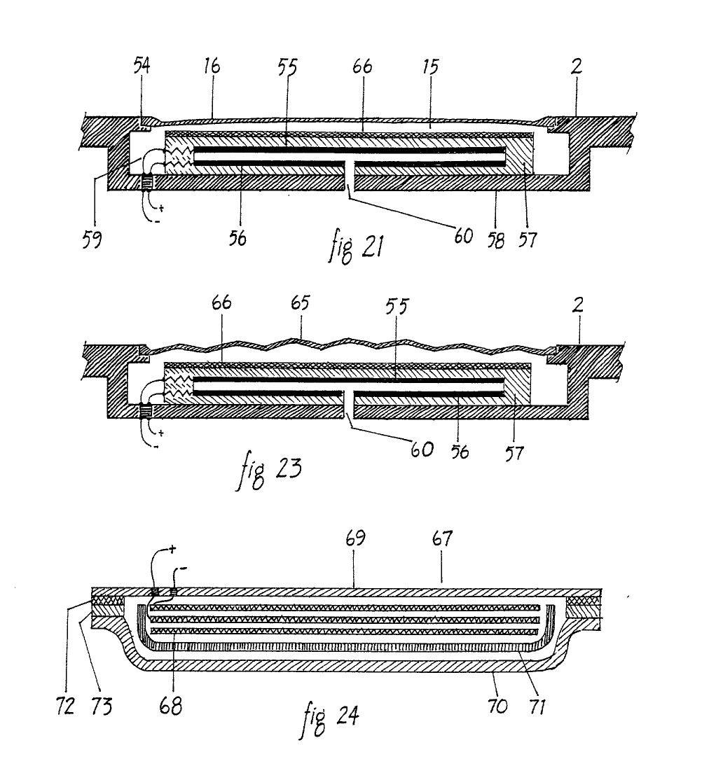

Once said oxide layer is substantially removed, or at least thinned, polymer layer 47 (preferably FEP) shown in FIG. 18 must be activated using, for example a corona treatment or a sodium-based chemical treatment, to create surface oxygen sites for bonding to the bare titanium surface layer 48. Said FEP layer 47 can be fusion bonded to titanium surface 48 at about 300° C. at light pressure for about 1-30 minutes, preferably about 5-10 minutes. It is important to note that high vacuum (i.e. about 10⁻⁵to 10⁻¹⁰ torr) must be maintained during said FEP heat bonding process. Once said heat bonding is complete the bonded parts can be cooled to room temperature and vacuum opened to normal atmosphere. FIG. 19 shows layer 47 directly heat bonded to titanium bottom plate 3. Although such direct bonding of FEP layer 47 to titanium bottom plate 3 is direct and elegant, the technical engineering details required to accomplish said bonding are complex. FIG. 20 illustrates the fusion of an FEP layer 43 (containing platinum conductor lines 44 and contact pads 45), similar to FIG. 15, heat bonded to FEP layer 47.

A yet further alternate embodiment for achieving a reliable fluid-impermeable bond between a polymer layer and titanium surface can be accomplished by first electroplating an approximately 1-25 micron thick gold layer onto the distal surface of the (titanium) bottom plate, or alternately, by placing an approximately 1-25 micron thick gold shim onto said bottom plate surface. Said gold plating (or gold shim) are then fusion bonded to said (titanium) surface by heating in a vacuum of at least about 10⁻⁴ torr to a few degrees above the melting point of gold for several minutes so as to create a thin intermetallic gold-titanium alloy, and then cooling to room temperature and breaking vacuum to atmosphere. This process creates a bonded layer to the distal titanium (oxide) surface layer of the bottom plate. Said deposited gold layer can now be bonded to a first polymer layer (containing a composite deposited layer 28, as depicted in FIG. 9) using a low temperature solder, such as tin or an alloy of tin, or an alloy containing one or more of, bismuth, indium, zinc, silver or gold. In an alternative embodiment, tin can be vacuum deposited onto the deposited gold layer.

Another aspect of the invention is the incorporation of a low profile manually activated safety on-off switch to allow the implantee to turn off the implanted device in case of device failure or simply as a need to disable the implanted device. Such a switch must be highly reliable, easily activated by the implantee, and robust to withstand external accidental blows to the skin overlaying said switch. FIG. 21 shows a cross-section of contact switch 15 incorporated into the proximal side of the implant enclosure which switch 15 is covered with a flexible membrane 16, preferably titanium (about 25-250 microns thick), welded at its circumference 54 to the proximal side of the (titanium) top plate 2. By manually pressing membrane 16 with a finger (see FIG. 22), the switch contacts 55 and 56 are closed, and reopened when the pressure is released. A non-conductive material such as a fluorocarbon or polyimide 57 acts to hold and electrically insulate said contacts 55 and 56 during the “open” position. A support plate 58 acts to mechanically support switch 15, with leads 59 from switch 15 electrically isolated from plate 58. Plate 58 is designed to withstand a high impact loading due to accidental blows to the skin overlaying membrane 16. A metal disc may be attached to the proximal side of the polymer 57 covering contact 55, which disc acts to provide mechanical support during switch activation. An optional hole 60 leads from within the enclosure of the implant to the gap between switch contacts 55 and 56. Said hole 60 provides for an optional air pressure equalization between said gap and the rest of the enclosure during switch activation. Said switch activation is illustrated by FIG. 22, which shows a finger 61 pushing against skin 62 on the proximal side of the implant, such action causing contacts 55 and 56 to touch, thereby causing the switch contacts to close, and reopen when the induced pressure is removed. FIG. 22 also shows the distal part of the preferred embodiment of the implant positioned against bone 63 to provide a reaction force against that induced by the pushing of finger 61.

An alternate embodiment to membrane 16 (shown in FIG. 21) is a “rippled” membrane 65 shown in FIG. 23, where said membrane 65 is preferably titanium, with such “rippling” providing enhanced flexibility to membrane 65 during activation to close contacts 55 and 56.

FIG. 24 is a sketch of a hermetically encapsulated rechargeable (secondary) battery contained within the housing of the implant, the location of the battery 67 being shown in FIGS. 1 and 22. The preferred embodiment of said battery is comprised of one or a plurality of stacked lithium type rechargeable (i.e. secondary) cells 68, as described in U.S. patent application Ser. No. 09/450,025, which cells are damaged by exposure to air and water vapor, and are therefore preferably hermetically encapsulated. Such encapsulation is comprised of an insulative base plate 69 (comprised of, for example, alumina), cover plate 70 (comprised of, for example, copper, tin, indium, bismuth, silver, nickel, titanium or alloys thereof), insulative film 71 (comprised of, for example, a fluorocarbon or a poyimide), with base 69 hermetically sealed to cover plate 70 at the perimeter using low temperature alloys. Preferably, such sealing is accomplished by firstly fusing a layer of a metal or alloy 72 (containing, for example, one or more of titanium, silver and or gold) to base plate 69, and fusing alloy layer 72 to cover plate 70 using a solder 73 (containing tin, indium, bismuth, lead, silver or zinc, or an alloy thereof).

In a final alternate embodiment, the implant is substantially coated with a bio-inert coating 74, such as parylene or silicone, as shown in FIG. 25. Such a coating 74 acts to provide a further protective seal to the overall integrity of the device.

The above descriptions have been intended to illustrate the preferred and alternative embodiments of the invention. It will be appreciated that modifications and adaptations to such embodiments may be practiced without departing from the scope of the invention, such scope being most properly defined by reference to this specification as a whole and to the following claims.

What is claimed is:

1. A low profile, hermetic, bio-compatible enclosure for subcutaneous implantation in humans where said enclosure comprises:
a top plate;
a bottom plate secured to said top plate to form said enclosure, said bottom plate having an aperture therein;
a non-conducting insert hermetically bonded to said bottom plate so as to fill said aperture, said insert including a plurality of passageways extending therethrough to an inside of said enclosure, said passageways being filled with an electrically conducting metal or alloy;
a first polymer layer bonded to said bottom plate or to said non-conducting insert or to both said bottom plate and said non-conducting insert.

2. The enclosure of claim 1 further comprising a second polymer layer containing conducting wires and bonding pads, where at least a part of said second polymer layer is bonded to said first polymer layer.

3. The enclosure of claim 2 wherein said bonding pads are connected to said conducting metal or alloy in said sealed passageways in said non-conducting insert.

4. The enclosure of claim 3 further comprising a bio-inert foil bonded to said second polymer layer for protection against abrasion or tearing.

5. The enclosure of claim 3 further comprising a third polymer layer bonded to said second polymer layer for additional mechanical support.

6. The enclosure of claim 5 further comprising a bio-inert foil bonded to said third polymer layer for protection against abrasion or tearing.

7. The enclosure of claim 4 or claim 6 wherein said foil is comprised of bio-inert glass, ceramic or metal.

8. The enclosure of claim 4 or claim 6 wherein said foil is comprised of titanium or an alloy of titanium.

9. The enclosure of claim 1 wherein said top plate and bottom plate are comprised one or more of a combination of bio-inert metal, glass or ceramic.

10. The enclosure of claim 1 wherein the top plate and bottom plate are comprised of titanium, or an alloy of titanium.

11. The enclosure of claim 1 wherein said insert is comprised of ceramic or glass.

12. The enclosure of claim 1 wherein said insert is comprised of alumina or sapphire.

13. The enclosure of claim 1 wherein said electrically conducting metal or alloy is comprised of titanium, silver, copper, gold, tin or any combination thereof.

14. The enclosure of claim 3 wherein said bonding pads are electrically connected to said electrically conducting metal or alloy in said passageways using a conductive epoxy or paste or a metal or alloy.

15. The enclosure of claim 14 wherein said metal or alloy is comprised of one or more of tin, bismuth, indium, zinc, silver or gold.

16. The enclosure of claim 2 wherein the bonding pads are comprised of platinum.

17. The enclosure of claim 1 wherein said insert has a substantially round, rectangular, square of oval shape.

18. The enclosure of claim 1 wherein said non-conducting insert is bonded to said bottom plate by means of silver, or an alloy of silver.

19. The enclosure of claim 1 wherein said non-conducting insert is bonded to said bottom plate by means of an active brazing alloy containing titanium.

20. The enclosure of claim 1 wherein said non-conducting insert is bonded to said bottom plate by means of gold, or an alloy of gold.

21. The enclosure of claim 1 wherein one side of said bottom plate has a retaining groove into which a polymer is molded, such that said molded polymer acts as an O-ring anchor within said bottom plate.

22. The enclosure of claim 21 further comprising a first polymer layer fused to said O-ring anchor, such fusion acting to create a strong mechanical anchor for said first polymer layer.

23. The enclosure of claim 1, 2 or 5 wherein said polymer layer(s) are comprised of one or more of the fluorocarbons, polypropylene, polyethylene, polyimide, or polyamide.

24. The enclosure of claim 23 where said polymer layers are comprised of the fluorocarbon FEP.

25. The enclosure of claim 1 where the side of said first polymer layer facing said bottom plate has been coated by bonding one or more metal layers thereto.

26. The enclosure of claim 25 wherein a first one of said metal layers is zirconium, tantalum, niobium, aluminum or tin.

27. The enclosure of claim 25 wherein a first one of said metal layers is titanium.

28. The enclosure of claim 26 or 27 wherein the surface of said first polymer layer has been chemically treated with a corona discharge or a sodium-based solution to make said surface more reactive.

29. The enclosure of claim 26 or 27 wherein additional metal layers are bonded to said first metal layer, said additional metal layers being comprised of one or more of niobium, gold and tin.

30. The enclosure of claim 1 wherein a side of said bottom plate or non-conducting insert has been coated by bonding one or more metal layers thereto.

31. The enclosure of claim 30 wherein said first metal layer is gold or an alloy of gold

32. The enclosure of claim 30 wherein said first metal layer is silver of an alloy of silver.

33. The enclosure of claim 30 wherein said first metal layer is an active brazing alloy containing titanium.

34. The enclosure of claim 27 where said titanium coated polymer surface is bonded to a side of said bottom plate or non-conducting insert using silicone.

35. The enclosure of claim 27 wherein said first metal layer is bonded to said bottom plate and non-conducting insert using an epoxy.

36. The enclosure of claim 31, 32 and 33 wherein the second of said metal layers is bonded to said first metal layer and is an alloy containing one or more of niobium, tin, bismuth, indium, zinc, silver or gold.

37. The enclosure of claim 1 wherein said first polymer layer has been coated by bonding a multiple metal layer thereto, said metal layer comprising a plurality of concentric round, oval or “wavy” rings.

38. The enclosure of claim 1 wherein said bottom plate has been coated by bonding a multiple metal layer thereto, said metal layer comprising a plurality of concentric “wavy” rings.

39. A method for fabricating a low profile hermetic, biocompatible enclosure for subcutaneous implantation in humans wherein said enclosure comprises:
a top plate;
a titanium bottom plate secured to said top plate to form said enclosure, said bottom plate having an aperture therein;
a non-conducting insert hermetically bonded to said titanium bottom plate so as to fill said aperture, said insert including a plurality of passageways extending therethrough to an inside of said enclosure, said passageways being filled with an electrically conducting metal or alloy;
a first polymer layer coated by bonding one or more metal layers thereto;
a said bottom plate whose distal side has been coated by bonding one or more metal layers thereto;
said method comprising bonding said metal- coated first polymer layer to said metal-coated bottom plate or non-conducting insert by fusing the multiple metal layers on the first polymer layer to the one or more metal layers on the bottom plate or non-conducting insert using a solder comprised of one or more of tin, bismuth, indium, zinc, silver or gold, or an alloy thereof.

40. A method for fabricating a low profile hermetic, biocompatible enclosure for subcutaneous implantation in humans wherein said enclosure comprises:
a titanium top plate;
a titanium bottom plate secured to said top plate to form said enclosure, said bottom plate having an aperture therein;
a non-conducting insert hermetically bonded to said titanium bottom plate so as to fill said aperture, said insert including a plurality of passageways extending therethrough to an inside of said enclosure, said passageways being filled an electrically conducting metal or alloy;
a first polymer layer coated by bonding one or more metal layers thereto, said metal layers comprising a plurality of concentric “wavy” rings;
said bottom plate being coated by bonding one or more metal layers thereto, said metal layers comprising a plurality of concentric “wavy” rings;
said method comprising bonding the metal-coated first polymer layer to the metal-coated bottom plate by bonding the concentric “wavy” rings on the first polymer layer to the concentric “wavy” rings on the bottom plate using solder comprised of tin, bismuth, indium, zinc, silver or gold, or alloys thereof.

41. A method for fabricating a low profile hermetic, bio-compatible enclosure for subcutaneous implantation in humans where said enclosure comprises:
a top plate;
a titanium bottom plate secured to said top plate to form said enclosure, said bottom plate having an aperture therein;
a non-conducting insert hermetically bonded to said bottom plate so as to fill said aperture, said insert including a plurality of passageways extending therethrough to an inside of said enclosure, said passageways being filled an electrically conducting metal or alloy;
said method comprising bonding a first polymer layer to said bottom plate by:
heating the titanium bottom plate to between 300 and 1,500° C., in a vacuum of about 10⁻⁵ to 10⁻¹⁰ torr so as to substantially remove the surface oxide layer from the titanium surface to create a highly reactive titanium surface;
subsequently cooling the titanium bottom plate to about 300° C., while maintaining said vacuum, and fusion bonding at about 300° C., under light pressure, said first polymer layer to the highly reactive titanium surface, and cooling to room temperature and breaking vacuum to normal atmosphere.

42. A method for fabricating a low profile hermetic, bio-compatible enclosure for subcutaneous implantation in humans where said enclosure comprises:
a top plate;
a titanium bottom plate secured to said top plate to form said enclosure, said bottom plate having an aperture therein;
a non-conducting insert hermetically bonded to said bottom plate so as to fill said aperture, said insert including a plurality of passageways extending therethrough to an inside of said enclosure, said passageways being filled an electrically conducting metal or alloy;
Said method comprising bonding a first polymer layer to said titanium bottom plate by:
chemically etching the titanium bottom plate with a dilute solution of HF (hydrofluoric acid) in a normal or an inert atmosphere so as to substantially thin the surface oxide layer at the titanium surface to create a more reactive titanium surface;
washing off any residual HF solution and drying the titanium bottom plate;
heating the titanium bottom plate to about 300° C., and fusion bonding at about 300° C., under light pressure, said first polymer layer to the titanium surface, and cooling to room temperature.

43. A method for fabricating a low profile hermetic, bio-compatible enclosure for subcutaneous implantation in humans where said enclosure comprises:
a top plate;
a titanium bottom plate secured to said top plate to form said enclosure, said bottom plate having an aperture therein;
a non-conducting insert hermetically bonded to said bottom plate so as to fill said aperture, said insert including a plurality of passageways extending therethrough to an inside of said enclosure, said passageways being filled an electrically conducting metal or alloy;
said method comprising bonding a first polymer layer to the titanium bottom plate or titanium-coated non-conducting insert by:
adding an approximately 1-25 micron thick coating of gold onto the surface of said bottom plate of said non-conducting insert;
bonding said gold layer to said titanium surface by heating in a vacuum of at least about 10⁻⁴ torr to a few degrees above the melting point of gold for several minutes so as to create a thin intermetallic gold-titanium alloy, and then cooling to room temperature and breaking vacuum to atmosphere;
vacuum depositing about 0.005 to 5 micron thick layer of titanium onto the surface of said first polymer layer, and while still maintaining vacuum, depositing a niobium layer about 0.2 to 5 microns thick, followed by a gold deposited layer about 0.2 to 5 microns thick, followed by a tin deposited layer;
bonding the deposited tin layer and the gold layer on the bottom plate or non-conducting insert, by using a solder containing one or more of tin, bismuth, indium, zinc, silver or gold, or alloys thereof.

44. The method of claim 39 where the surface of said first polymer layer is comprised of FEP and has been treated with a corona discharge to activate the surface.

45. The method of claim 39 where the surface of said first polymer layer is comprised of FEP and has been treated with a sodium-based chemical to activate the surface.

46. The method of claim 39 where a second polymer layer is fusion bonded to said first polymer layer by heating in a vacuum.

47. The method of claim 46 where a third polymer layer is fusion bonded to said second polymer layer by heating in a vacuum.

48. The enclosure of claim 1 where a safety (or panic off) mechanically actuated switch is contained on said inside of said enclosure.

49. The enclosure of claim 48 where said switch is comprised of a flexible membrane, welded at the circumference to said top plate.

50. The enclosure of claim 49 where said membrane is rippled to provide enhanced flexibility to said membrane during activation to close the switch contacts.

51. The enclosure of claims 49 and 50 where the membrane, when manually pressed, causes switch contacts to close, and reopen when the induced pressure is removed.

52. A low profile hermetic, bio-compatible enclosure for subcutaneous implantation in humans where said enclosure comprises:
a top plate;
a bottom plate secured to said top plate to form said enclosure, said bottom plate having an aperture therein;
a non-conducting insert hermetically bonded to said bottom plate so as to fill said aperture, said insert including a plurality of passageways extending therethrough to an inside of said enclosure, said passageways being filled an electrically conducting metal or alloy;
a first polymer layer bonded to said bottom plate or non-conducting insert; and,
a secondary battery contained within said enclosure.

53. The enclosure of claim 52 where said battery is comprised of one or a plurality of stacked lithium type rechargeable (secondary) cells.

54. The enclosure of claim 52 where said battery is hermetically encapsulated, such encapsulation comprised of an insulative base plate, and a cover plate hermetically sealed to said base plate at the perimeter using a solder containing one or more of tin, indium, lead, silver, zinc and bismuth, or an alloy thereof.

55. The enclosure of claim 1 where said enclosure is substantially encapsulated with a bio-inert coating of parylene or silicone.

56. The enclosure of claim 1 where the overall thickness of said enclosure is between 2 and 7 mm.

57. The enclosure of claim 1 where the diameter of said enclosure of the invention is between 3 and 50 mm.
